(19) United States
(12) Patent Application Publication
(10) Pub. No.: US 2026/0060708 A1
(43) Pub. Date: Mar. 5, 2026

(54) THROMBECTOMY APPARATUS

(71) Applicant: Boston Scientific Scimed, Inc., Maple Grove, MN (US)

(72) Inventors: Joseph Stanley Czyscon, Elk River, MN (US); Wonjun Hwang, Plymouth, MN (US); Derek Kenneth Larson, Golden Valley, MN (US)

(73) Assignee: Boston Scientific Scimed, Inc., Maple Grove, MN (US)

(21) Appl. No.: 19/317,230

(22) Filed: Sep. 3, 2025

Related U.S. Application Data
(60) Provisional application No. 63/690,441, filed on Sep. 4, 2024.

(51) Int. Cl. A61B 17/221; A61M 25/00

(52) U.S. Cl. CPC A61B 17/221; A61M 25/0029; A61B 2017/2215; A61M 2025/004

(57) ABSTRACT

A medical assembly for ingesting a thrombus or embolus includes an outer catheter and a middle catheter that is slidingly disposed within the outer catheter. A funnel is coupled to the middle catheter. The funnel is adapted to fit within an outer catheter lumen when the middle catheter is moved proximally relative to the outer catheter and to extend out of the outer catheter lumen when the middle catheter is moved distally relative to the outer catheter. An inner catheter is slidingly disposed within the middle catheter lumen. An ingestion sleeve is coupled to the inner catheter and passes over the funnel periphery and extends proximally therefrom. Moving the middle catheter distally with respect to the inner catheter causes the ingestion sleeve to slide over the funnel periphery, thereby pulling the thrombus or embolus through the funnel and into the middle catheter lumen.

### CROSS-REFERENCE TO RELATED APPLICATIONS

This application claims the benefit of priority under 35 U.S.C. § 119 of U.S. Provisional Application No. 63/690,441, filed Sep. 4, 2024, the entire disclosure of which is hereby incorporated by reference.

### TECHNICAL FIELD

The present disclosure pertains to medical devices, and methods for manufacturing and using medical devices. More particularly, the present disclosure pertains to medical devices, methods, and systems for performing thrombectomy procedures.

### BACKGROUND

Removal of material from within a vessel or chamber is often useful. For example, removal of tissue such as blood clots from within a vasculature may improve patient conditions and quality of life. Clot removal may be beneficial or even necessary to improve patient outcomes. For example, in the peripheral vasculature, interventions and procedures can reduce the need for an amputation by 80 percent. The ultimate goal of any technique to treat these conditions is to remove the blockage and to restore patency, quickly, safely, and cost effectively.

Devices for mechanically removing material, including thrombus material, from within a lumen of the vessel may include an inverting tube for removing material from a body lumen, such as for removing a clot from a blood vessel (e.g., thrombectomy devices), are disclosed and described in each of U.S. Pat. Nos. 10,271,864, 10,835,234, 10,561,431, 10,842,513 and 11,253,291. The described apparatuses may face challenges when removing large amounts of material which may require multiple attempts to remove, and/or when removing softer clot material, which may be difficult to grip. In some cases, it may be difficult to completely remove clot material from against the wall of the vessel. Thus, there is an ongoing need for devices, including thrombectomy devices, that can remove tissue, and particularly large and/or soft materials, from within a body lumen.

### SUMMARY

This disclosure provides design, material, manufacturing method, and use alternatives for medical devices. An example may be found in a medical assembly for ingesting a thrombus or embolus. The medical assembly includes an outer catheter defining an outer catheter lumen and a middle catheter that is slidingly disposed within the outer catheter lumen. The middle catheter includes a distal region and defines a middle catheter lumen. A funnel is coupled to the distal region of the middle catheter and includes a funnel periphery. The funnel is adapted to fit within the outer catheter lumen when the middle catheter is moved proximally relative to the outer catheter and to extend out of the outer catheter lumen when the middle catheter is moved distally relative to the outer catheter. An inner catheter is slidingly disposed within the middle catheter lumen, the inner catheter including a distal region. An ingestion sleeve is coupled to the distal region of the inner catheter and is adapted to pass over the funnel periphery and extend proximally therefrom within the middle catheter lumen. Moving the middle catheter distally with respect to the inner catheter causes the ingestion sleeve to slide over the funnel periphery, thereby pulling the thrombus or embolus through the funnel and into the middle catheter lumen.

Alternatively or additionally, the outer catheter may have a 16 French or less outer diameter.

Alternatively or additionally, the middle catheter and the outer catheter may be adapted to be moved together in a distal direction relative to the inner catheter when ingesting the thrombus or embolus.

Alternatively or additionally, the outer catheter may further include an outer catheter proximal hub and the middle catheter may further include a middle catheter proximal hub. Distal movement of the middle catheter relative to the outer catheter may be limited by the middle catheter proximal hub engaging the outer catheter proximal hub.

Alternatively or additionally, the outer catheter proximal hub may be adapted to limit proximal travel of the middle catheter relative to the outer catheter.

Alternatively or additionally, the ingestion sleeve may have a pre-ingestion configuration in which the ingestion sleeve extends a first distance proximally of the funnel periphery.

Alternatively or additionally, the ingestion sleeve may have a post-ingestion configuration in which the ingestion sleeve extends a second distance, less than the first distance, proximally of the funnel periphery.

Alternatively or additionally, the ingestion sleeve may further include a radiopaque marker.

Alternatively or additionally, an annular void may be defined between an outer diameter of the middle catheter and an inner diameter of the outer catheter lumen that is adapted for transporting contrast medium to a position proximate the funnel.

Another example may be found in a medical kit for ingesting a thrombus or embolus. The medical kit includes an outer assembly and an inner assembly. The outer assembly includes an outer catheter that defines an outer catheter lumen and a middle catheter that is slidingly disposed within the outer catheter lumen. The middle catheter includes a distal region and defining a middle catheter lumen. The outer assembly includes a funnel that includes a funnel periphery. The middle catheter is coupled to the outer catheter with the funnel extending distally from the outer catheter lumen. The inner assembly includes an inner catheter that is slidingly disposed within the middle catheter lumen, the inner catheter including a distal region, and an ingestion sleeve that is coupled to the distal region of the inner catheter and is adapted to pass over the funnel periphery and extend proximally therefrom within the middle catheter lumen. Advancing the outer assembly distally while holding the inner assembly in place relative to the thrombus causes the ingestion sleeve to slide over the funnel periphery as the funnel moves distally, thereby pulling the thrombus or embolus through the funnel and into the middle catheter lumen.

Alternatively or additionally, the outer catheter may have a 16 French or less outer diameter.

Alternatively or additionally, the outer catheter may further include an outer catheter proximal hub and the middle catheter may further include a middle catheter proximal hub. Distal movement of the middle catheter relative to the outer catheter is limited by the middle catheter proximal hub engaging the outer catheter proximal hub.

Alternatively or additionally, the outer catheter proximal hub may be adapted to limit proximal travel of the middle catheter relative to the outer catheter.

Alternatively or additionally, the ingestion sleeve may have a pre-ingestion configuration in which the ingestion sleeve extends a first distance proximally of the funnel periphery.

Alternatively or additionally, the ingestion sleeve may have a post-ingestion configuration in which the ingestion sleeve extends a second distance, less than the first distance, proximally of the funnel periphery.

Another example may be found in a method of removing a thrombus or embolus from within a patient's vasculature. The method includes advancing an assembly through the vasculature to a position proximate the thrombus or embolus, the assembly including an outer catheter, a middle catheter slidingly disposed within the outer catheter and bearing a funnel, and an inner catheter slidingly disposed within the middle catheter and bearing an ingestion sleeve extending over a periphery of the funnel. The inner catheter is held in position relative to the thrombus or embolus and the middle catheter and the outer catheter are advanced distally relative to the thrombus or embolus, thereby causing the ingestion sleeve to pull the thrombus or embolus through the funnel and into the middle catheter. The method includes moving the middle catheter proximally in order to withdraw the funnel into the outer catheter and withdrawing the assembly from the patient.

Alternatively or additionally, the method may further include viewing under fluoroscopy in order to determine when the funnel has exhausted its travel relative to the ingestion sleeve.

Alternatively or additionally, the method may further include subsequently advancing a second assembly through the vasculature to a position proximate the thrombus or embolus and repeating the holding, advancing and withdrawing steps in order to remove more of the thrombus or embolus.

Alternatively or additionally, the middle catheter may be locked in position with the funnel extending distally from the outer catheter when the assembly is advanced through the vasculature.

Alternatively or additionally, the method may further include unlocking the middle catheter from the outer catheter prior to withdrawing the funnel into the outer catheter.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The disclosure may be more completely understood in consideration of the following detailed description in connection with the accompanying drawings, in which:

FIG. 1 is a schematic view of an illustrative medical assembly for performing thrombectomy, with the medical assembly configured for advancement through the vasculature to reach a target thrombus or embolus;

FIG. 1A is a cross-sectional view taken along the line 1A-1A of FIG. 1;

FIG. 2 is a schematic view of the illustrative medical assembly of FIG. 1, with the medical assembly configured to remove the thrombus or embolus;

FIG. 3 is a schematic view of the illustrative medical assembly of FIG. 1, after having removed the thrombus or embolus;

FIG. 4 is a side view of an illustrative ingestion sleeve and funnel forming part of the illustrative medical assembly of FIG. 1; and

FIGS. 5, 6, 7 and 8 are views of the illustrative medical assembly of FIG. 1, demonstrating a step by step process for removing a thrombus or embolus.

It should be understood, however, that the intention is not to limit the invention to the particular embodiments described. On the contrary, the intention is to cover all modifications, equivalents, and alternatives falling within the spirit and scope of the disclosure.

### DETAILED DESCRIPTION

The following description should be read with reference to the drawings, which are not necessarily to scale, wherein like reference numerals indicate like elements throughout the several views. The detailed description and drawings are intended to illustrate but not limit the present disclosure. Those skilled in the art will recognize that the various elements described and/or shown may be arranged in various combinations and configurations without departing from the scope of the disclosure. The detailed description and drawings illustrate example embodiments of the disclosure. However, in the interest of clarity and ease of understanding, while every feature and/or element may not be shown in each drawing, the feature(s) and/or element(s) may be understood to be present regardless, unless otherwise specified.

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that in order to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For simplicity and clarity purposes, not all elements of the present disclosure are necessarily shown in each figure or discussed in detail below. However, it will be understood that the following discussion may apply equally to any and/or all of the components for which there are more than one, unless explicitly stated to the contrary. Additionally, not all instances of some elements or features may be shown in each figure for clarity.

Relative terms such as “proximal”, “distal”, “advance”, “retract”, variants thereof, and the like, may be generally considered with respect to the positioning, direction, and/or operation of various elements relative to a user/operator/manipulator of the device, wherein “proximal” and “retract” indicate or refer to closer to or toward the user and “distal” and “advance” indicate or refer to farther from or away from the user. In some instances, the terms “proximal” and “distal” may be arbitrarily assigned in an effort to facilitate understanding of the disclosure, and such instances will be readily apparent to the skilled artisan. Other relative terms, such as “upstream”, “downstream”, “inflow”, and “outflow” refer to a direction of fluid flow within a lumen, such as a body lumen, a blood vessel, or within a device. Still other relative terms, such as “axial”, “circumferential”, “longitudinal”, “lateral”, “radial”, etc. and/or variants thereof generally refer to direction and/or orientation relative to a central longitudinal axis of the disclosed structure or device.

The terms “monolithic” and “unitary” shall generally refer to an element or elements made from or consisting of a single structure or base unit/element. A monolithic and/or unitary element shall exclude structure and/or features made by assembling or otherwise joining multiple discrete elements together.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to use the particular feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is exemplary only. In some embodiments, alterations of and deviations from previously used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

Thrombectomy is a process whereby a thrombus or embolus, or clot, is removed from within a blood vessel. In some cases, a thrombus forms in location while an embolus is a thrombus that has moved to a new location. In some cases, all pulmonary embolisms are caused by an embolus that originated as a thrombus within the deep venous system. Thrombectomy may be performed using a medical assembly for ingesting a thrombus or embolus. The medical assembly includes an outer catheter defining an outer catheter lumen and a middle catheter that is slidingly disposed within the outer catheter lumen. The middle catheter includes a distal region and defines a middle catheter lumen. A funnel including a funnel periphery is coupled to the distal region of the middle catheter. The funnel is adapted to fit within the outer catheter lumen when the middle catheter is moved proximally relative to the outer catheter and to extend out of the outer catheter lumen when the middle catheter is moved distally relative to the outer catheter. An inner catheter includes a distal region and is slidingly disposed within the middle catheter lumen. An ingestion sleeve is coupled to the distal region of the inner catheter and is adapted to pass over the funnel periphery and extend proximally therefrom within the middle catheter lumen. Moving the middle catheter distally with respect to the inner catheter causes the ingestion sleeve to slide over the funnel periphery, thereby pulling the thrombus or embolus through the funnel and into the middle catheter lumen. In some cases, the outer catheter may have a 16 French (or smaller) outer diameter. In some cases, the outer catheter may have a 14 French, or even a 12 French, outer diameter. In some cases, the medical assembly may be larger, with an outer catheter having up to a 24 French outer diameter.

In some cases, the middle catheter and the outer catheter may be adapted to be moved together in a distal direction relative to the inner catheter when ingesting the thrombus or embolus. In some cases, the outer catheter may further include an outer catheter proximal hub and the middle catheter may further include a middle catheter proximal hub. Distal movement of the middle catheter relative to the outer catheter may be limited by the middle catheter proximal hub engaging the outer catheter proximal hub. The outer catheter proximal hub may be adapted to limit proximal travel of the middle catheter relative to the outer catheter.

In some cases, the ingestion sleeve may have a pre-ingestion configuration in which the ingestion sleeve extends a first distance proximally of the funnel periphery. The ingestion sleeve may have a post-ingestion configuration in which the ingestion sleeve extends a second distance, less than the first distance, proximally of the funnel periphery. In some cases, the ingestion sleeve may further include a radiopaque marker. In some cases, an annular void that may be defined between an outer diameter of the middle catheter and an inner diameter of the outer catheter lumen may be adapted for transporting contrast medium to a position proximate the funnel.

In some instances, thrombectomy may be performed using a medical kit for ingesting a thrombus or embolus. The medical kit includes an outer assembly and an inner assembly. The outer assembly includes an outer catheter that defines an outer catheter lumen and a middle catheter that is slidingly disposed within the outer catheter lumen. The middle catheter includes a distal region and defining a middle catheter lumen. A funnel including a funnel periphery is coupled to the distal region of the middle catheter. The middle catheter is coupled to the outer catheter with the funnel extending distally from the outer catheter lumen. The inner assembly includes an inner catheter that includes a distal region and is slidingly disposed within the middle catheter lumen. An ingestion sleeve is coupled to the distal region of the inner catheter and is adapted to pass over the funnel periphery and extend proximally therefrom within the middle catheter lumen. Advancing the outer assembly distally while holding the inner assembly in place relative to the thrombus or embolus causes the ingestion sleeve to slide over the funnel periphery as the funnel moves distally, thereby pulling the thrombus or embolus through the funnel and into the middle catheter lumen. In some cases, the outer catheter may have a 16 French (or smaller) outer diameter. In some cases, the outer catheter may have a 14 French, or even a 12 French, outer diameter. In some cases, the medical assembly may be larger, with an outer catheter having up to a 24 French outer diameter.

In some cases, the outer catheter may further include an outer catheter proximal hub and the middle catheter may further include a middle catheter proximal hub. Distal movement of the middle catheter relative to the outer catheter may be limited by the middle catheter proximal hub engaging the outer catheter proximal hub. In some cases, the outer catheter proximal hub may be adapted to limit proximal travel of the middle catheter relative to the outer catheter. The ingestion sleeve may have a pre-ingestion configuration in which the ingestion sleeve extends a first distance proximally of the funnel periphery and a post-ingestion configuration in which the ingestion sleeve extends a second distance, less than the first distance, proximally of the funnel periphery.

In some instances, thrombectomy may be performed via a method of removing a thrombus or embolus from within a patient's vasculature. The method includes advancing an assembly through the vasculature to a position proximate the thrombus or embolus, the assembly including an outer catheter, a middle catheter slidingly disposed within the outer catheter and bearing a funnel, and an inner catheter slidingly disposed within the middle catheter and bearing an ingestion sleeve extending over a periphery of the funnel. The inner catheter is held in position relative to the thrombus or embolus and the middle catheter and the outer catheter are advanced distally relative to the thrombus or embolus, thereby causing the ingestion sleeve to pull the thrombus or embolus through the funnel and into the middle catheter. The middle catheter is moved proximally in order to withdraw the funnel into the outer catheter. The assembly is then withdrawn from the patient.

In some cases, the method may further include viewing under fluoroscopy in order to determine when the funnel has exhausted its travel relative to the ingestion sleeve. In some cases, the method may further include subsequently advancing a second assembly through the vasculature to a position proximate the thrombus or embolus and repeating the holding, advancing and withdrawing steps in order to remove more of the thrombus or embolus. In some cases, the middle catheter may be locked in position with the funnel extending distally from the outer catheter when the assembly is advanced through the vasculature. In some cases, the method may further include unlocking the middle catheter from the outer catheter prior to withdrawing the funnel into the outer catheter.

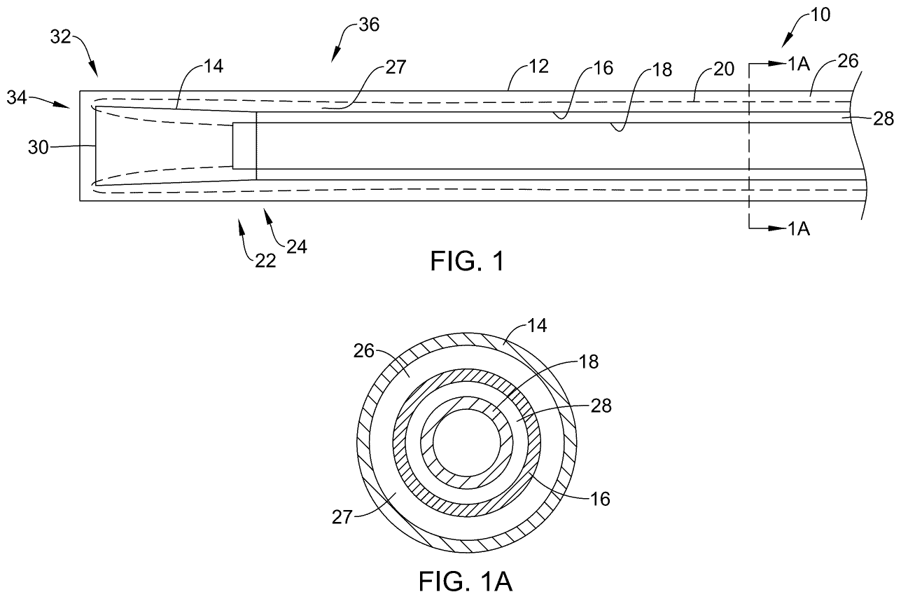

FIG. 1 is a schematic view of an illustrative medical assembly 10 that is adapted for ingesting a thrombus or embolus within a patient's blood vessel. In some cases, the illustrative medical assembly 10 may provide benefits over known thrombectomy apparatuses by incorporating the access/aspiration sheath, maintaining guidewire access, improving device repositionability by including an ability to re-sheathe the funnel and improving case of use for the medical assembly 10 for the physician using the medical assembly 10. The medical assembly 10 provides for a reduced preparation time ahead of use. The medical assembly 10 provides a separate lumen for injecting contrast medium. In some cases, the medical assembly 10 may provide at least some of these benefits through a shorter overall system length relative to known thrombectomy apparatuses.

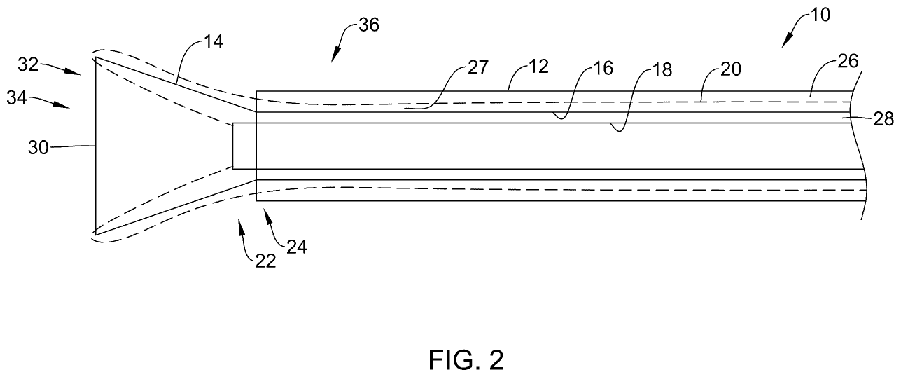

FIG. 1 shows the medical assembly 10 in a configuration for advancement through the patient's vasculature, with an outer catheter 12 and a funnel 14 remaining disposed within the outer catheter 12. FIG. 2 shows the medical assembly 10 in a configuration in which the medical assembly 10 is ready to ingest a thrombus or embolus, with the funnel 14 extending distally from the outer catheter 12. The funnel 14 also extends distally from a middle catheter 16. An inner catheter 18 extends within the middle catheter 16 and includes an ingestion sleeve 20 that extends from the inner catheter 18. In some cases, the ingestion sleeve 20 extends from a distal region 22 of the inner catheter 18. In some cases, the funnel 14 extends from a distal region 24 of the middle catheter 16. The outer catheter 12 defines an outer catheter lumen 26 and the middle catheter 16 defines a middle catheter lumen 28. As shown, the middle catheter 18 may be slidingly disposed within the outer catheter lumen 26. The inner catheter 18 may be slidingly disposed within the middle catheter lumen 28. The ingestion sleeve 20 may be considered as extending from the distal region 22, over a funnel periphery 30, and proximally within the outer catheter lumen 26. More specifically, the ingestion sleeve 20 may be considered as extending proximally within an annular void 27 formed between an inner diameter of the outer catheter lumen 26 and an outer diameter of the middle catheter 16, for example. The annular void 27 may be considered as the volume within the outer catheter lumen 26 less the volume consumed by the middle catheter 16 extending within the outer catheter lumen 26. The annular void 27 may also be seen in FIG. 1A, which is a cross-sectional view taken along the lines 1A-1A of FIG. 1. In some cases, the funnel 14 may be considered as being adapted to fit within the outer catheter lumen 26 when the middle catheter 16 is moved proximally relative to the outer catheter 12 and to extend out of the outer catheter lumen 26 when the middle catheter 16 is moved distally relative to the outer catheter 12.

In some cases, a thrombus or embolus may be ingested by holding the inner catheter 18 in position relative to the thrombus or embolus and advancing the middle catheter 16 forward. As the middle catheter 16 (and hence the funnel 14) moves distally relative to the ingestion sleeve 20, the ingestion sleeve 20 is caused to invert over a funnel periphery 30 as the funnel 14 moves distally relative to the inner catheter 18 and the ingestion sleeve 20. The ingestion sleeve 20 inverting over the funnel periphery 30 causes the thrombus or embolus to be pulled into the funnel 14 and into the middle catheter lumen 28. In some cases, the outer catheter 12 and the middle catheter 16 may both be moved distally relative to the inner catheter 18 and the ingestion sleeve 20 in order to ingest the thrombus or embolus. In some cases, as will be discussed, the outer catheter 12 and the middle catheter 16 may be moved together in unison. In some cases, the outer catheter 12 and the middle catheter 16 may be adapted to be locked together in order to make it easier to move the outer catheter 12 and the middle catheter 16 distally as a single unit. In some cases, the middle catheter 16 may be adapted to subsequently be unlocked from the outer catheter 12 such that the middle catheter 16 may be moved proximally relative to the outer catheter 12 in order to re-sheathe the funnel 14.

In some cases, the outer catheter 12 and the middle catheter 16, including the funnel 14, may be considered as being an outer assembly 32 and the inner catheter 18, including the ingestion sleeve 20, may be considered as being an inner assembly 34. Together, the outer assembly 32 and the inner assembly 34 may be considered as being the medical assembly 10. In some cases, the outer assembly 32 and the inner assembly 34 may together be considered as being a medical kit 36 that may be used for removing a thrombus or embolus from a patient's blood vessel. In some cases, advancing the outer assembly 32 distally while holding the inner assembly 34 in place relative to the thrombus or embolus may cause the ingestion sleeve 20 to slide over the funnel periphery 30 as the funnel 14 moves distally, thereby pulling the thrombus or embolus through the funnel 14 and into the middle catheter lumen 28. In some cases, the outer catheter 12 and the middle catheter 16 may be adapted to be locked together to form the outer assembly 32. However, in some cases, the middle catheter 16 may be adapted to subsequently be unlocked from the outer catheter 12 such that the middle catheter 16 may be moved proximally relative to the outer catheter 12 in order to re-sheathe the funnel 14.

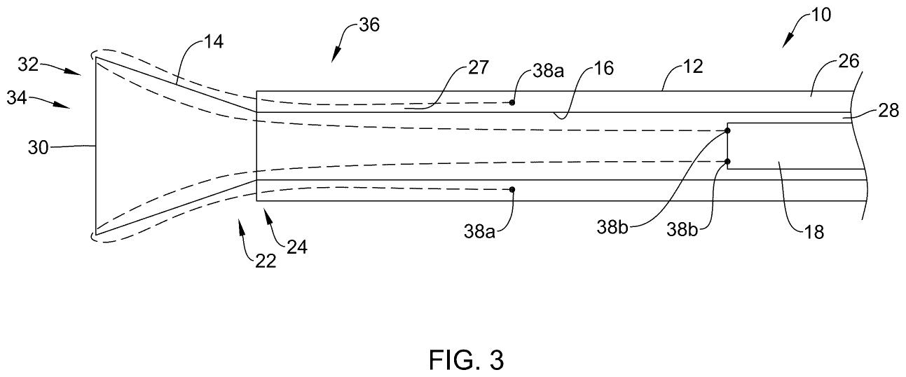

FIG. 3 shows the medical assembly 10 (or the medical kit 36) after a thrombus or embolus or at least a portion of a thrombus or embolus has been ingested through the funnel 14 and into the middle catheter lumen 28. The outer assembly 32 has been advanced distally relative to the inner assembly 34. The distal region 22 of the inner catheter 18 is positioned proximally of the distal region 24 of the middle catheter 16, for example. This distance may be a function of the overall length of the ingestion sleeve 20, allowing for a certain length of the ingestion sleeve 20 to remain within the outer catheter lumen 26. In some cases, the ingestion sleeve 20 may be allowed to be pulled completely out of the outer catheter lumen 26. In some cases, the ingestion sleeve 20 may have a pre-ingestion configuration (as shown for example in FIG. 2) in which the ingestion sleeve 20 extends a first distance proximally of the funnel periphery 30. The ingestion sleeve 20 may extend proximally within the outer catheter lumen 26, for example. The ingestion sleeve 20 may have a post-ingestion configuration (as shown for example in FIG. 3) in which the ingestion sleeve 20 extends a second distance, less than the first distance, proximally of the funnel periphery 30.

In some instances, the middle catheter 16 may be dimensioned, relative to the outer catheter 12, such that the middle catheter 16 may be advanced distally relative to the outer catheter 12 a distance sufficient to allow the funnel 14 to extend distally from the outer catheter 12. In some cases, the middle catheter 16 may have a length that allows the funnel 14 to extend out of the outer catheter 12 in its entirety, but to not extend further distally than that. In some cases, the middle catheter 16 may be allowed to translate relative to the outer catheter 12 a distance that allows the funnel 14 to extend out of the outer catheter 12 and to move back into the outer catheter lumen 26, but is not allowed to translate beyond that, i.e., the funnel 14 can extend distally from the outer catheter 12, but the middle catheter 16 cannot, and the funnel 14 can withdraw into the outer catheter lumen 26 with the funnel periphery 30 remaining close to the distal end of the outer catheter lumen 26.

In some cases, lengths are relative. A longer outer catheter 12 necessitates a longer middle catheter 16, for example. The inner catheter 18 has a length that allows the ingestion sleeve 20 to extend over the funnel periphery 30 and into the outer catheter lumen 26. How far the outer catheter 12 and the middle catheter 16 (or the outer assembly 32) is able to move distally relative to the inner catheter 18 and the ingestion sleeve 20 (or the inner assembly 34) is a function of how long the ingestion sleeve 20 is. It will be appreciated that the overall dimensions of the medical assembly 10 (or the medical kit 36) will depend on the patient in which the medical assembly 10 (or the medical kit 36) will be used. The medical assembly 10 (or the medical kit 36) may be made in several distinct sizes, such as small and large, or perhaps small, medium and large.

The outer catheter 12 may have a length ranging from 75 centimeters (29.5 inches) to 110 centimeters (43.3 inches) while the middle catheter 16 may correspondingly have a length ranging from 87 centimeters (34.3 inches) to 122 centimeters (48.0 inches). The inner catheter 18 may have a length ranging from 102 centimeters (40.2 inches) to 137 centimeters (53.9 inches) while the ingestion sleeve 20 may have a length ranging from 60 centimeters (23.6 inches) to 80 centimeters (31.5 inches), for example.

For a small size, the outer catheter 12 may have a length of 75 centimeters (29.5 inches), the middle catheter 16 may have a length of 87 centimeters (34.3 inches), the inner catheter 18 may have a length of 102 centimeters (40.2 inches) and the ingestion sleeve 20 may have a length of 60 centimeters (23.6 inches). For a middle size, the outer catheter 12 may have a length of 90 centimeters (35.4 inches), the middle catheter 16 may have a length of 102 centimeters (40.2 inches), the inner catheter 18 may have a length of 117 centimeters (46.1 inches) and the ingestion sleeve 20 may have a length of 70 centimeters (27.6 inches). For a large size, the outer catheter 12 may have a length of 110 centimeters (43.3 inches), the middle catheter 16 may have a length of 122 centimeters (48.0 inches), the inner catheter 18 may have a length of 137 centimeters (53.9 inches) and the ingestion sleeve 20 may have a length of 80 centimeters (31.5 inches).

In some cases, the outer catheter 12 may have a 16 French or less outer diameter. In some cases, the outer catheter 12 may have a 14 French outer diameter, or even a 12 French outer diameter. It will be appreciated that the middle catheter 16 and the inner catheter 18 may be correspondingly sized. When the outer catheter 12 has a 16 French outer diameter, the middle catheter 16 and the inner catheter 18 will have larger corresponding outer diameters relative to when the outer catheter 12 has a 14 French outer diameter, or even a 12 French outer diameter. As an example, for a 16 French system, the outer catheter 12 may have a 16 French outer diameter, the middle catheter 16 may have a 14 French outer diameter and the inner catheter 18 may have a 4 French outer diameter. For a 14 French system, the outer catheter 12 may have a 14 French outer diameter, the middle catheter 16 may have a 10 French outer diameter and the inner catheter 18 may have a 4 French outer diameter. For a 12 French system, the outer catheter 12 may have a 12 French outer diameter, the middle catheter 16 may have a 8 French outer diameter and the inner catheter 18 may have a 4 French outer diameter.

In some cases, a process of removing a thrombus may be viewed fluoroscopically. In some cases, the ingestion sleeve 20 may include one or more radiopaque markers that can provide an indication of how much relative travel between the middle catheter 16 and the inner catheter 18 is allowed. The radiopaque markers may be provided in a variety of different locations. As an example, the ingestion sleeve 20 may include one, two, three or more radiopaque markers 38a that are located at or near a free end of the ingestion sleeve 20. As another example, the ingestion sleeve 20 may include one, two, three or more radiopaque markers 38b that are located at or near an opposing attached end of the ingestion sleeve 20. Radiopaque markers may alternatively or additionally be secured within the distal region 22 of the inner catheter 18. The radiopaque markers may be made of any suitable material that produces a discernible image during fluoroscopy.

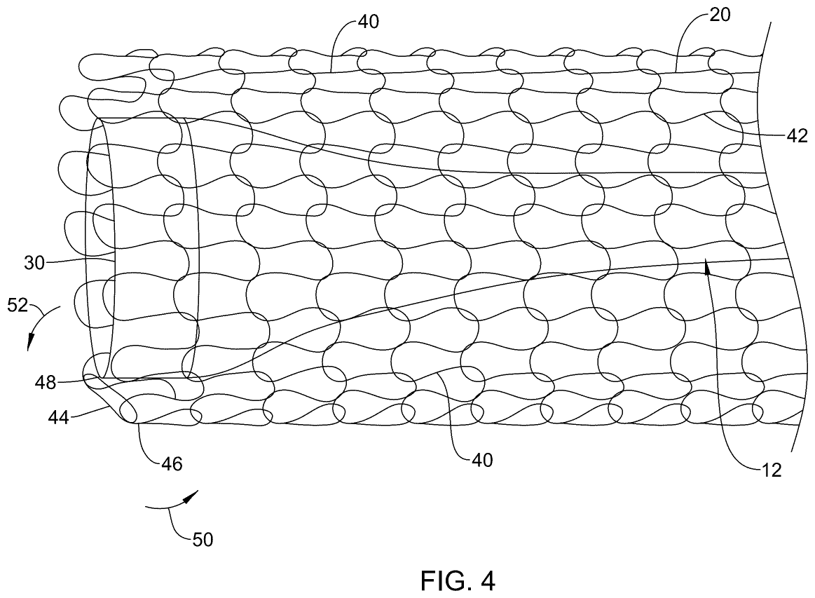

FIG. 4 is a schematic view of the ingestion sleeve 20 disposed over the funnel 14, and inverting over the funnel periphery 30. In some instances, as shown, the ingestion sleeve 20 may be formed of a plurality of loops 40. In some cases, the ingestion sleeve 20 may be formed by knitting one or more filaments 42 into the plurality of loops 40. A variety of different knitting patterns may be utilized, for example. It will be appreciated that the ingestion sleeve 20 inverts itself as the individual loops 40 travel over the funnel periphery 30. With particular attention to a specific loop 44, the specific loop 44 has a leading edge 46 and a trailing edge 48. As the specific loop 44 pivots around the funnel periphery 30, the leading edge 46 moves in a direction indicated by an arrow 50 while the trailing edge 46 moves in a direction indicated by an arrow 52. Accordingly, as each of the loops 40 pivot around the funnel periphery 30, each loop 40 will temporarily extend radially outwardly as the loop 40 pivots. Otherwise, each of the loops 40 will lay flat.

As the middle catheter 16 (and hence the funnel 14) moves distally relative to the ingestion sleeve 20, an axial compressive force may be applied to the funnel 14 (and particularly the funnel periphery 30) by the ingestion sleeve 20. In some cases, the funnel 14 may be adapted to resist or otherwise accommodate this axial compressive force such that the funnel 14 is able to retain its tapered shape. In some cases, the funnel 14 may include a braided pattern that is adapted to resist any applied axial compressive forces. Additional details regarding examples of construction patterns for the funnel 14 may be found in U.S. Provisional Application Ser. Nos. 63/540,599; 63/540,608; and 63/540,611; each of which were filed Sep. 26, 2023 and each of which were entitled “THROMBECTOMY APPARATUS”; each of these applications are herein incorporated by reference in their entireties.

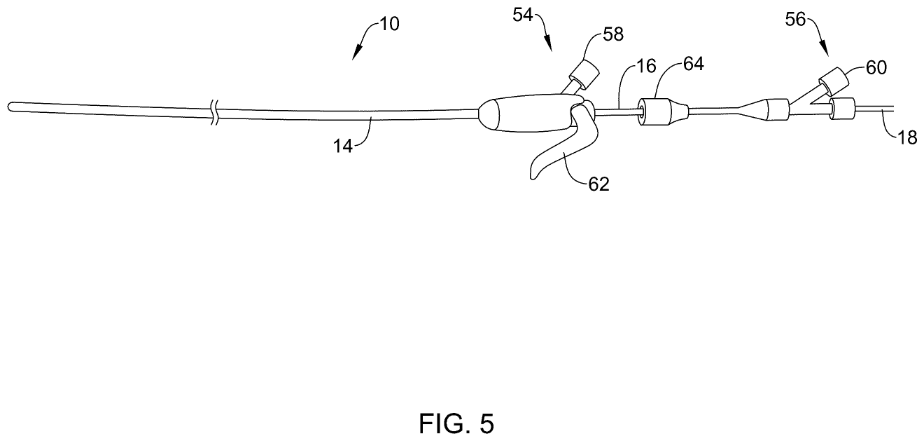

FIGS. 5, 6, 7 and 8 are views of the medical assembly 10 (or the medical kit 36), demonstrating a step by step process for removing a thrombus or embolus. FIG. 5 shows the medical assembly 10, including the outer catheter 12, the middle catheter 16 and the inner catheter 18. Not visible in this view are the funnel 14 and the ingestion sleeve 20. Prior to use, the medical assembly 10 may be removed from packaging and each of the outer catheter 12, the middle catheter 16 and the inner catheter 18 are flushed with saline in order to remove any possible air that may be present within any of the components of the medical assembly 10. In some cases, the outer catheter 12 includes an outer catheter proximal hub 54 and the middle catheter 16 includes a middle catheter proximal hub 56. The outer catheter proximal hub 54 and the middle catheter proximal hub 56 may each be used to flush the outer catheter lumen 26 and the middle catheter lumen 28, respectively. As shown, the outer catheter proximal hub 54 includes a flushing port 58 and the middle catheter proximal hub 56 includes a flushing port 60. Once flushed, the medical assembly 10 may be advanced through the patient's vasculature until a thrombus is reached.

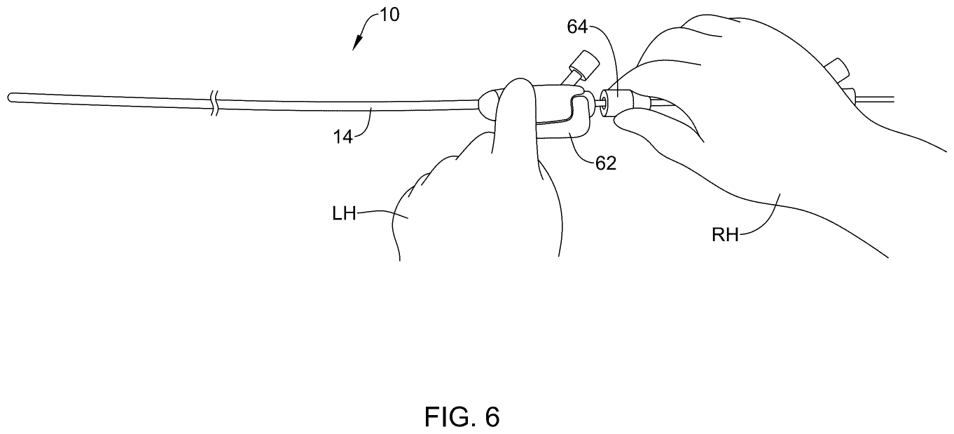

In some cases, as shown, the outer catheter proximal hub 54 also includes a lever 62. The lever 62 may be actuated to lock the middle catheter 16 in a particular axial position relative to the outer catheter 12. As shown for example in FIG. 6, a physician or other medical professional may hold the outer catheter 12 in their left hand LH while they advance the middle catheter 16 distally relative to the outer catheter 12 by holding the middle catheter 16 in their right hand RH. In some cases, the middle catheter 16 includes a stop member 64 that the user may grasp when urging the middle catheter 16 in a distal direction relative to the outer catheter 12. As will be appreciated, the stop member 64 will limit distal travel of the middle catheter 16 relative to the outer catheter 12. When the stop member 64 contacts the outer catheter proximal hub 54, this corresponds to the funnel 14 (not visible) having just extended distally out of the outer catheter lumen 26. The user may engage the lever 62 in order to secure the middle catheter 16 in position relative to the outer catheter 12. In some cases, the outer catheter 12 and/or the middle catheter 16 may include other features that limit how far the middle catheter 16 may be withdrawn proximally relative to the outer catheter 12. In some cases, the middle catheter proximal hub 56 may include features that limit relative proximal travel. In some cases, the middle catheter 16 itself may include a feature that limits proximal travel of the middle catheter 16 by interacting with the outer catheter proximal hub 54.

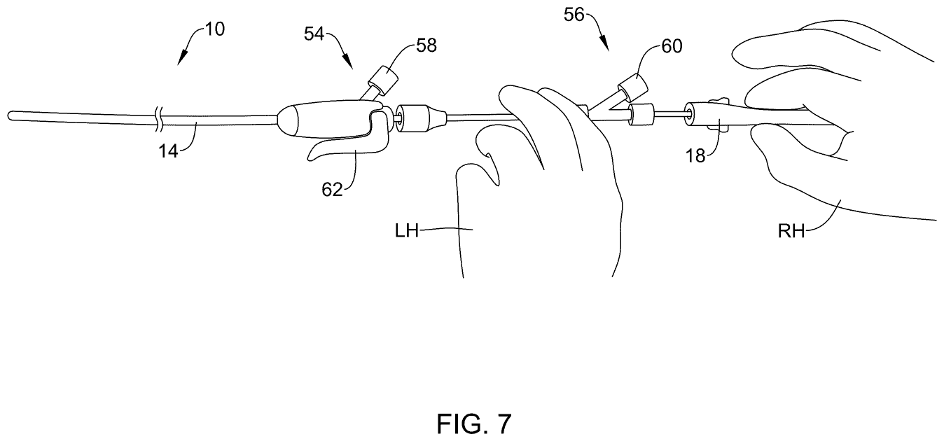

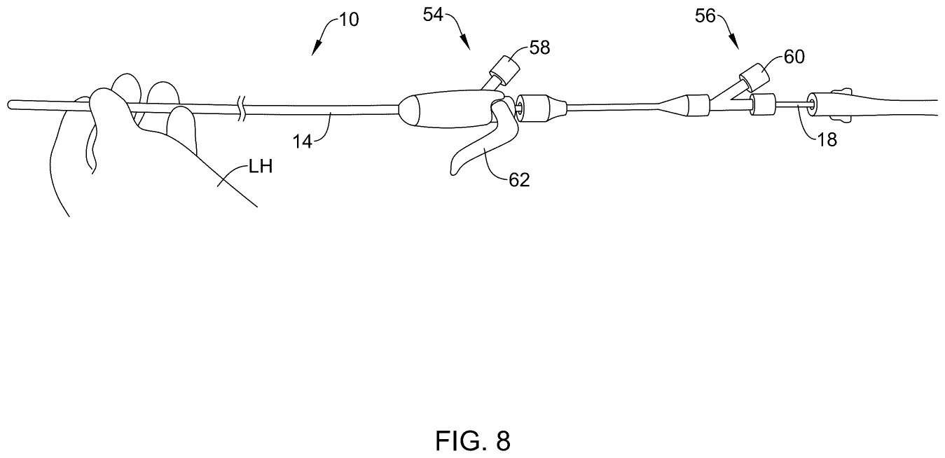

Next, as shown in FIG. 7, the user may hold the inner catheter 18 in their right hand RH while they grasp the middle catheter proximal hub 56 in their left hand LH and urge the middle catheter 16 (and the outer catheter 12) in a distal direction relative to the inner catheter 18. As can be seen by comparing FIG. 7 to FIG. 5, the lever 62 is in a locked position. As the funnel 14 moves distally relative to the ingestion sleeve 20 as a result of the middle catheter 16 moving distally relative to the inner catheter 18 and hence the ingestion sleeve 20, the ingestion sleeve 20 urges the thrombus or embolus into the middle catheter lumen 28. Once the ingestion sleeve 20 has been fully utilized (as may be determined under fluoroscopy, for example), the user may withdraw the middle catheter 16 and the outer catheter 12 proximally relative to the inner catheter 18, thereby returning the medical assembly 10 to a configuration in which the medical assembly 10 may be withdrawn from the patient, as shown in FIG. 8. In some cases, this may include unlocking the middle catheter 16 from the outer catheter 12 so that the funnel 14 may be withdrawn back into the outer catheter lumen 26. If the thrombus or embolus has not been completely removed, a second medical assembly 10 may be prepared and used in the same manner in order to fully remove the thrombus or embolus, or to remove another thrombus or embolus at a different location.

The materials that can be used for the various components of the devices and various elements thereof disclosed herein may include those commonly associated with medical devices. In some instances, the medical devices, and/or components thereof, may be made from a metal, metal alloy, polymer (some examples of which are disclosed below), a metal-polymer composite, ceramics, combinations thereof, and the like, or other suitable material.

Some examples of suitable polymers may include polytetrafluorocthylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), MARLEX® high-density polyethylene, MARLEX® low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro (propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, polyurethane silicone copolymers (for example, ElastEon® from Aortech Biomaterials or ChronoSil® from AdvanSource Biomaterials), biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

Some examples of suitable metals and metal alloys include stainless steel, such as 304V, 304L, and 316LV stainless steel; mild steel; nickel-titanium alloy such as linear-clastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276R, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; platinum; palladium; gold; combinations thereof; or any other suitable material.

In at least some instances, portions or all of the medical devices described herein, and/or components thereof, may also be doped with, made of, or otherwise include a radiopaque material. Radiopaque materials are understood to be materials capable of producing a relatively bright image on a fluoroscopy screen or another imaging technique during a medical procedure. This relatively bright image aids the user of the apparatus in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the apparatus to achieve the same result.

In some instances, a degree of Magnetic Resonance Imaging (MRI) compatibility is imparted into the medical devices and/or other elements disclosed herein. For example, the medical devices, and/or components or portions thereof, may be made of a material that does not substantially distort the image and create substantial artifacts (e.g., gaps in the image). Certain ferromagnetic materials, for example, may not be suitable because they may create artifacts in an MRI image. The medical assembly 10, or portions thereof, may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

In some instances, the medical devices and/or other elements disclosed herein may include and/or be treated with a suitable therapeutic agent. Some examples of suitable therapeutic agents may include anti-thrombogenic agents (such as heparin, heparin derivatives, urokinase, and PPack (dextrophenylalanine proline arginine chloromethylketone)); anti-proliferative agents (such as enoxaparin, angiopeptin, monoclonal antibodies capable of blocking smooth muscle cell proliferation, hirudin, and acetylsalicylic acid); anti-inflammatory agents (such as dexamethasone, prednisolone, corticosterone, budesonide, estrogen, sulfasalazine, and mesalamine); antincoplastic/antiproliferative/anti-mitotic agents (such as paclitaxel, 5-fluorouracil, cisplatin, vinblastine, vincristine, epothilones, endostatin, angiostatin and thymidine kinase inhibitors); anesthetic agents (such as lidocaine, bupivacaine, and ropivacaine); anti-coagulants (such as D-Phe-Pro-Arg chloromethyl keton, an RGD peptide-containing compound, heparin, anti-thrombin compounds, platelet receptor antagonists, anti-thrombin antibodies, anti-platelet receptor antibodies, aspirin, prostaglandin inhibitors, platelet inhibitors, and tick antiplatelet peptides); vascular cell growth promoters (such as growth factor inhibitors, growth factor receptor antagonists, transcriptional activators, and translational promoters); vascular cell growth inhibitors (such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin); cholesterol-lowering agents; vasodilating agents; and agents which interfere with endogenous vasoactive mechanisms.

Having thus described several illustrative examples of the present disclosure, those of skill in the art will readily appreciate that yet other examples may be made and used within the scope of the claims hereto attached. It will be understood, however, that this disclosure is, in many respects, only illustrative. Changes may be made in details, particularly in matters of shape, size, arrangement of parts, and exclusion and order of steps, without exceeding the scope of the disclosure. The disclosure's scope is, of course, defined in the language in which the appended claims are expressed.

What is claimed is:

1. A medical assembly for ingesting a thrombus or embolus, the medical assembly comprising:
an outer catheter defining an outer catheter lumen;
a middle catheter slidingly disposed within the outer catheter lumen, the middle catheter including a distal region and defining a middle catheter lumen;
a funnel coupled to the distal region of the middle catheter, the funnel including a funnel periphery, the funnel adapted to fit within the outer catheter lumen when the middle catheter is moved proximally relative to the outer catheter and to extend out of the outer catheter lumen when the middle catheter is moved distally relative to the outer catheter;
an inner catheter slidingly disposed within the middle catheter lumen, the inner catheter including a distal region; and
an ingestion sleeve coupled to the distal region of the inner catheter and adapted to pass over the funnel periphery and extend proximally therefrom within the middle catheter lumen;
wherein moving the middle catheter distally with respect to the inner catheter causes the ingestion sleeve to slide over the funnel periphery, thereby pulling the thrombus or embolus through the funnel and into the middle catheter lumen.

2. The medical assembly of claim 1, wherein the outer catheter has a 16 French or less outer diameter.

3. The medical assembly of claim 1, wherein the middle catheter and the outer catheter are adapted to be moved together in a distal direction relative to the inner catheter when ingesting the thrombus or embolus.

4. The medical assembly of claim 1, wherein:
the outer catheter further comprises an outer catheter proximal hub;
the middle catheter further comprises a middle catheter proximal hub; and
distal movement of the middle catheter relative to the outer catheter is limited by the middle catheter proximal hub engaging the outer catheter proximal hub.

5. The medical assembly of claim 4, wherein the outer catheter proximal hub is adapted to limit proximal travel of the middle catheter relative to the outer catheter.

6. The medical assembly of claim 1, wherein the ingestion sleeve has a pre-ingestion configuration in which the ingestion sleeve extends a first distance proximally of the funnel periphery.

7. The medical assembly of claim 6, wherein the ingestion sleeve has a post-ingestion configuration in which the ingestion sleeve extends a second distance, less than the first distance, proximally of the funnel periphery.

8. The medical assembly of claim 1, wherein the ingestion sleeve further comprises a radiopaque marker.

9. The medical assembly of claim 1, wherein:
an annular void is defined between an outer diameter of the middle catheter and an inner diameter of the outer catheter lumen; and
the annular void is adapted for transporting contrast medium to a position proximate the funnel.

10. A medical kit for ingesting a thrombus or embolus, the medical kit comprising:
an outer assembly including:
an outer catheter defining an outer catheter lumen;
a middle catheter slidingly disposed within the outer catheter lumen, the middle catheter including a distal region and defining a middle catheter lumen;
a funnel coupled to the distal region of the middle catheter, the funnel including a funnel periphery;
the middle catheter coupled to the outer catheter with the funnel extending distally from the outer catheter lumen; and
an inner assembly including:
an inner catheter slidingly disposed within the middle catheter lumen, the inner catheter including a distal region; and
an ingestion sleeve coupled to the distal region of the inner catheter and adapted to pass over the funnel periphery and extend proximally therefrom within the middle catheter lumen;
wherein advancing the outer assembly distally while holding the inner assembly in place relative to the thrombus or embolus causes the ingestion sleeve to slide over the funnel periphery as the funnel moves distally, thereby pulling the thrombus or embolus through the funnel and into the middle catheter lumen.

11. The medical kit of claim 10, wherein the outer catheter has a 16 French or less outer diameter.

12. The medical kit of claim 10, wherein:
the outer catheter further comprises an outer catheter proximal hub;
the middle catheter further comprises a middle catheter proximal hub; and
distal movement of the middle catheter relative to the outer catheter is limited by the middle catheter proximal hub engaging the outer catheter proximal hub.

13. The medical kit of claim 12, wherein the outer catheter proximal hub is adapted to limit proximal travel of the middle catheter relative to the outer catheter.

14. The medical assembly of claim 10, wherein the ingestion sleeve has a pre-ingestion configuration in which the ingestion sleeve extends a first distance proximally of the funnel periphery.

15. The medical assembly of claim 14, wherein the ingestion sleeve has a post-ingestion configuration in which the ingestion sleeve extends a second distance, less than the first distance, proximally of the funnel periphery.

16. A method of removing a thrombus or embolus from within a patient's vasculature, the method comprising:
advancing an assembly through the vasculature to a position proximate the thrombus or embolus, the assembly including an outer catheter, a middle catheter slidingly disposed within the outer catheter and bearing a funnel, and an inner catheter slidingly disposed within the middle catheter and bearing an ingestion sleeve extending over a periphery of the funnel;
holding the inner catheter in position relative to the thrombus or embolus;
advancing the middle catheter and the outer catheter distally relative to the thrombus or embolus, thereby causing the ingestion sleeve to pull the thrombus or embolus through the funnel and into the middle catheter;
moving the middle catheter proximally in order to withdraw the funnel into the outer catheter; and
withdrawing the assembly from the patient.

17. The method of claim 16, further comprising viewing under fluoroscopy in order to determine when the funnel has exhausted its travel relative to the ingestion sleeve.

18. The method of claim 17, further comprising subsequently advancing a second assembly through the vasculature to a position proximate the thrombus or embolus and repeating the holding, advancing and withdrawing steps in order to remove more of the thrombus or embolus.

19. The method of claim 10, wherein the middle catheter is locked in position with the funnel extending distally from the outer catheter when the assembly is advanced through the vasculature.

20. The method of claim 19, further comprising unlocking the middle catheter from the outer catheter prior to withdrawing the funnel into the outer catheter.
